(19) United States
(12) Patent Application Publication
(10) Pub. No.: US 2020/0268415 A1
(43) Pub. Date: Aug. 27, 2020

(54) MULTI HEAD FOOT GRINDER

(71) Applicant: PROTV DEVELOPMENT INC., Taipei City (TW)

(72) Inventor: Jack TANG, NEW TAIPEI CITY (TW)

(21) Appl. No.: 16/378,275

(22) Filed: Apr. 8, 2019

(30) Foreign Application Priority Data
Feb. 27, 2019 (TW) 108202534

(51) Int. Cl. A61B 17/54

(52) U.S. Cl. CPC A61B 17/54; A61B 2017/00367

(57) ABSTRACT

A multi head foot grinder includes a housing having a grip and a front frame at the front side of the grip, a rotary rack rotatably mounted on the front frame, a plurality of grinding heads mounted in the rotary rack, a driving device mounted in the housing, a transmission mechanism mounted on the front frame and the rotary rack and coupled to the driving device and the grinding head that is located at the front end of the front frame and drivable by the driving device to rotate the coupled grinding head, and a locking knob mounted on the front frame and movable between a locking position and a release position.

### BACKGROUND OF THE INVENTION

### 1. Field of the Invention

The present invention relates to foot grinders and more particularly, to a multi head foot grinder that can switch the grinding head.

### 2. Description of the Related Art

Taiwan Patent D167834 discloses a shape design of a foot grinder. When this foot grinder is used, the front-sided grinding head is rotated to remove the dead skin or calluses from the sole of the foot, achieving the effect of beautifying the foot.

However, conventional foot grinders have only one grinding head, so only a single grinding roughness can be provided. Because each person has different dead skin sizes on the soles of the feet, the fineness of the grinding needs to be different. Therefore, if a grinding head with a different roughness is needed, multiple grinding heads with different roughness should be purchased for selective use. However, in this case, it is necessary to prepare a plurality of grinding heads for backup, which causes a problem of preservation, and the original grinding head needs to be removed when replacing, and then replaced with another grinding head, which is inconvenient to use.

### SUMMARY OF THE INVENTION

The present invention has been accomplished under the circumstances in view. It is the main object of the present invention to provide a multi head foot grinder, which has multiple grinding heads, which can be simply shifted so that the user can save the inconvenience of saving and replacing the grinding heads.

To achieve this and other objects of the present invention, a multi head foot grinder comprises a housing, a rotary rack, a plurality of grinding heads, a driving device, a transmission mechanism and a locking knob. The housing has a grip, and a front frame located on a front side of the grip. The rotary rack is rotatably mounted on the front frame. The grinding heads have an elongated shape, each comprising a pivot that has two opposite ends thereof pivotally mounted in the rotary rack such that when the rotary rack is rotated, one of the grinding heads is shifted to the front end of the front frame. The driving device is mounted in the housing. The transmission mechanism is mounted on the front frame and the rotary rack and coupled to the driving device and the grinding head that is shifted to the front end of the front frame. The transmission mechanism is drivable by the driving device to rotate the coupled grinding head. The locking knob is mounted on the front frame and movable between a locking position and a release position. The locking knob has a locking rod. The locking rod interferes with the rotary rack to prohibit the rotary rack from rotation when the locking knob is disposed in the locking position. The locking rod is released from the rotary rack for allowing the rotary rack to be rotated by an external force when the locking knob is disposed in the release position.

In this way, the invention can have multiple grinding heads arranged on one device for easy switching, so that the user can save the inconvenience of saving and replacing the grinding head, and solve the problems faced by the prior art design.

Other advantages and features of the present invention will be fully understood by reference to the following specification in conjunction with the accompanying drawings, in which like reference signs denote like components of structure.

### BRIEF DESCRIPTION OF THE DRAWINGS

FIG. 1 is an elevational view of a multi head foot grinder in accordance with the present invention.

FIG. 2 is an exploded view of the multi head foot grinder in accordance with the present invention.

FIG. 3 is similar to FIG. 2 when viewed from another angle.

FIG. 4 is an elevational view of a part of the present invention, illustrating the arrangement of the transmission mechanism.

FIG. 5 is similar to FIG. 4 when viewed from another angle.

FIG. 6 is an elevational view of a part of the present invention, illustrating the structure of the locking knob.

FIG. 7 is an exploded view of a part of FIG. 6 where the gear is excluded.

FIG. 8 is an elevational view of a part of the present invention, illustrating the structure of the switch button where the front arms of the front frame are excluded.

FIG. 9A is a schematic operational view of the present invention, illustrating the locking knob in the locking position.

FIG. 9B illustrates the locking rod in the locking position.

FIG. 10A is a schematic operational view of the present invention, illustrating the locking knob in the release position and the rotary rack rotated through a predetermined angle.

FIG. 10B illustrates the locking rod in the release position.

### DETAILED DESCRIPTION OF THE INVENTION

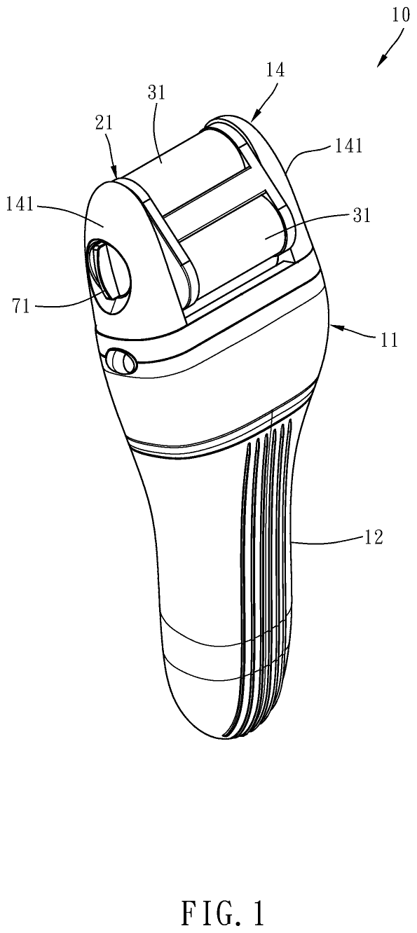

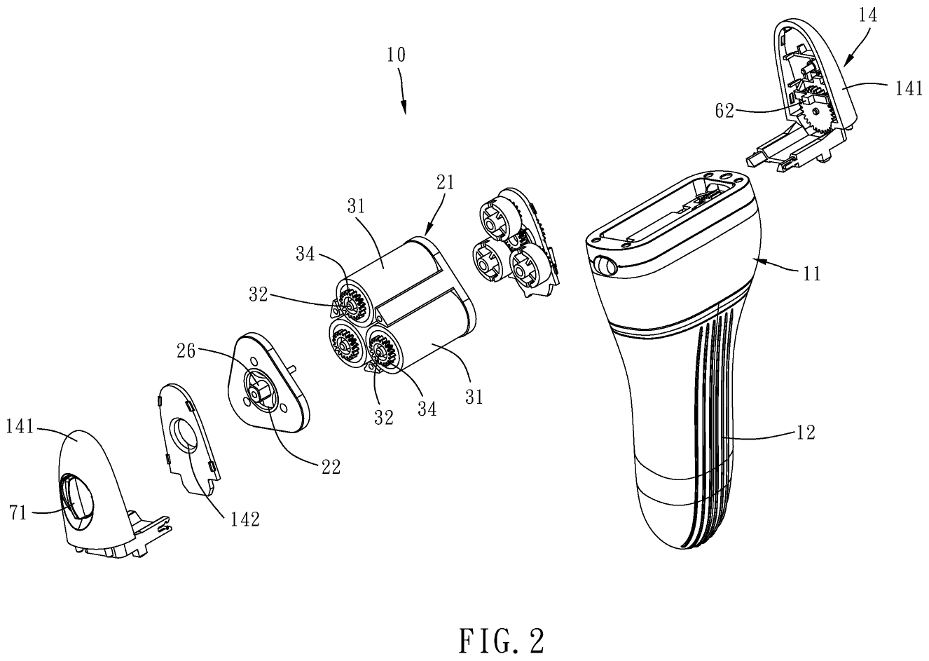

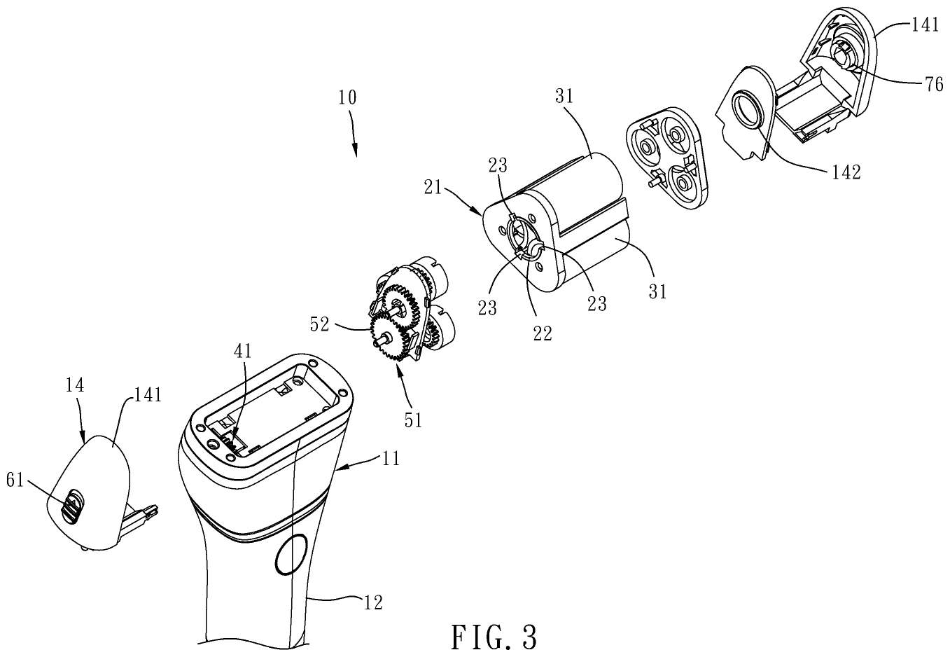

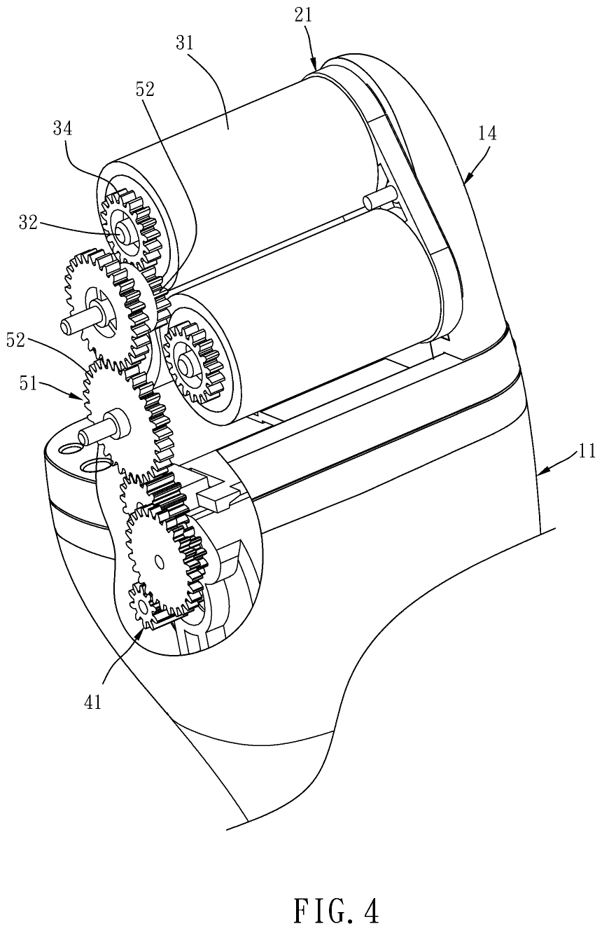

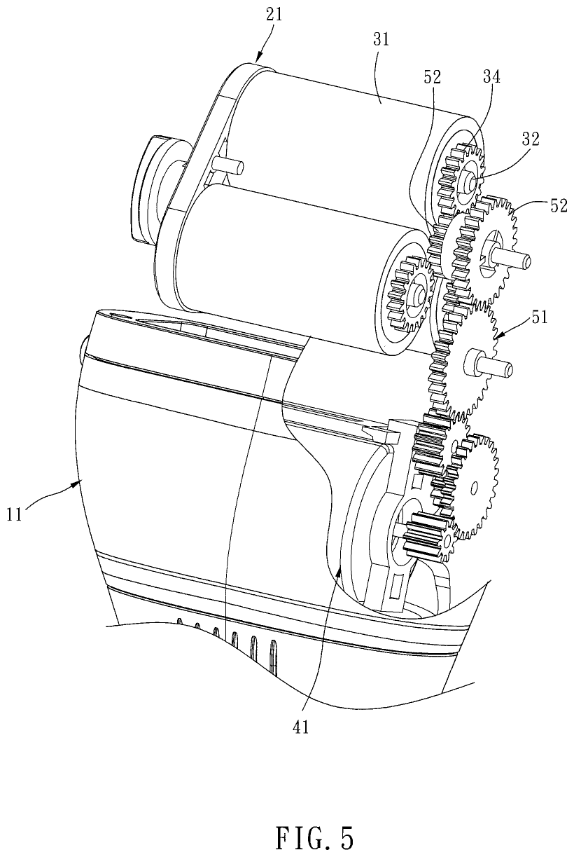

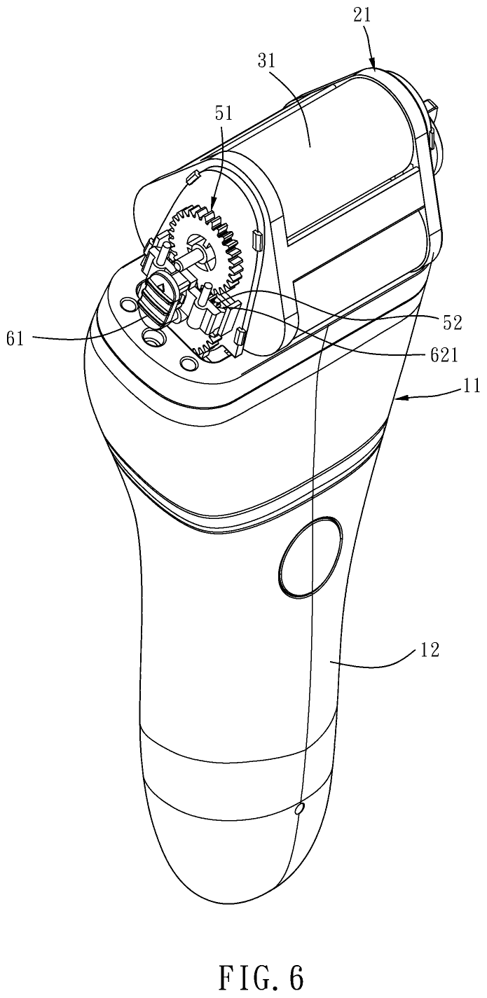

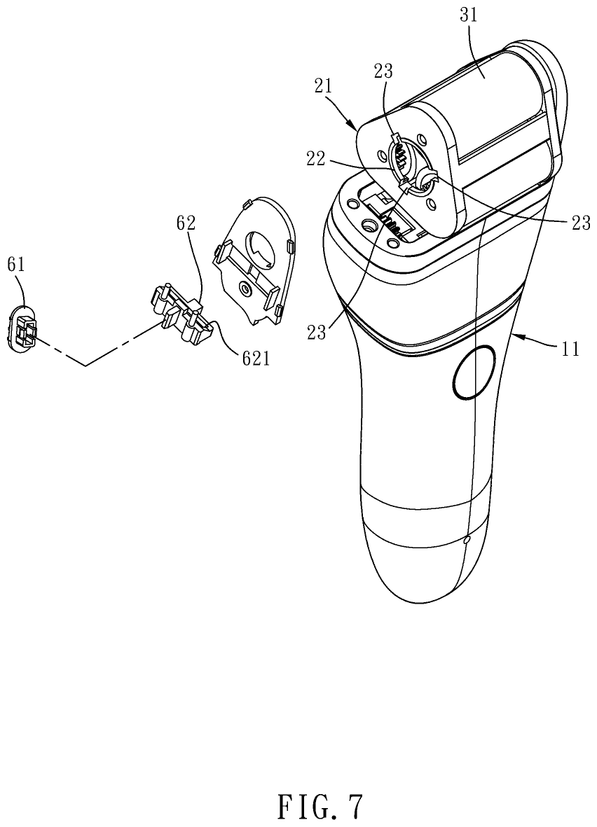

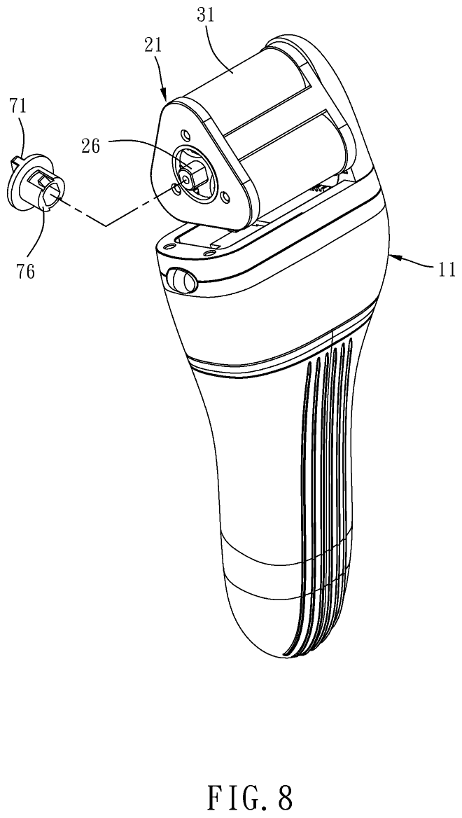

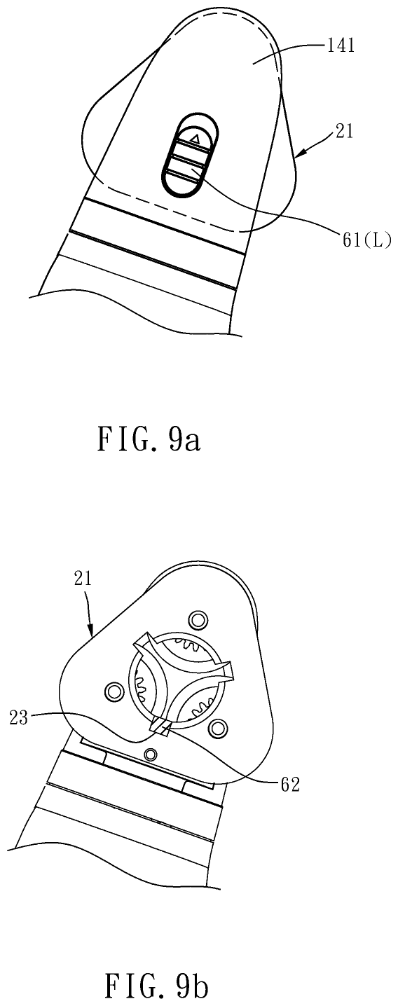

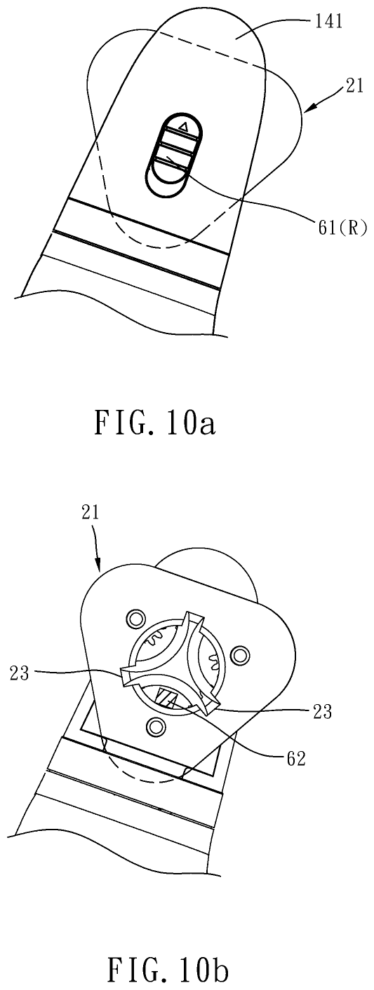

Referring to FIGS. 1-10B, a multi head foot grinder 10 in accordance with the present invention is shown. The multi head foot grinder 10 comprises a housing 11, a rotary rack 21, a plurality of grinding heads 31, a driving device 41, a transmission mechanism 51 and a locking knob 61.

The housing 11 comprises a grip 12, and a front frame 14 located at a front side of the grip 12. The front frame 14 has two front arms 141.

The rotary rack 21 is rotatably mounted to the front frame 14. In actual implementation, the front frame 14 has two circular ribs 142 respectively located on the two front arms 141 to face toward each other, and the rotary rack 21 has two circular holes 22 respectively located on two opposite sides thereof, further, the rotation axis of the rotary rack 21 (this is a virtual axis, not a solid structure) is also located in the circular holes 22. By means of inserting the circular ribs 142 into the respective circular holes 22, the rotary rack 21 is rotatably coupled to the front frame 14.

The multiple grinding heads 31 are elongated. In this embodiment, three grinding heads 31 are taken as an example. Each grinding head 31 has a pivot 32 that has two opposite ends thereof pivotally mounted in the rotary rack 21. When the rotary rack 21 is rotated, one of the grinding heads 31 is shifted to the front end of the front frame 14. Further, each grinding head 31 has a gear 34 mounted on one end of the pivot 32 thereof.

The driving device 41 is mounted in the housing 11. In this embodiment, the driving device 41 is a motor.

In this embodiment, the transmission mechanism 51 consists of a plurality of gears 52. These gears 52 are mounted on the front frame 14 and the rotary rack 21 and meshed with the gear 34 of one of the grinding heads 31 at the front frame 14 and drivable to rotate by the driving device 41. In the arrangement, the gears 52 of the transmission mechanism 51 can be maintained in a fixed position by the gear engagement relationship, so that only the gear 34 of the grinding head 31 located at the front end of the front frame 14 will be engaged with the gears 52 of the transmission with mechanism 51, i.e., only the grinding head 31 located at the front end of the front frame 14 will be rotated by the driving device 41. Since the rotating rack 21 is rotated, no matter which grinding head 31 is at the forefront, the position is the same, so it is inevitable that gear 34 of the grinding head 31 located at the front end of the front frame 14 can be engaged with the 52 of the transmission mechanism 51, and can be rotated by the driving device 41.

The locking knob 61 is mounted in the front frame 14 and movable between a locking position L and a release position R. The locking knob 61 has a locking rod 62. The rotary rack 21 further has a plurality of grooves 23 spaced around the border edge of the circular hole 22. These grooves 23 are spatially connected to the circular hole 22, and each of the grooves 23 corresponds to one respective grinding head 31. When the locking knob 61 is in the locking position L, the locking rod 62 is engaged into one groove 23 and interferes with the rotary rack 21 so that it cannot rotate. When the locking knob 61 is in the release position R, the locking rod 62 is disposed in the circular hole 22 and is not located in anyone of the grooves 23 and does not interfere with the rotary rack 21 so that the rotary rack 21 can be driven to rotate by an external force. In addition, when the transmission mechanism 51 and the locking rod 62 are located on the same side of the rotary rack 21, the locking rod 62 is arranged to have an accommodation space 621 and accommodate the gears 52 of the transmission mechanism 51, thus It is possible to perform the locking/release action without interfering with the transmission mechanism 51.

In the case that the rotary rack 21 is rotatable, the user can directly operate the rotary rack 21 by fingering the plural grinding heads 31. However, since the grinding head 31 is used for rubbing the soles of the feet, based on the hygiene considerations, a switch button 71 is provided for allowing the user to rotate the rotary rack 21 without touching the grinding heads 31.

The switch button 71 is disposed on the front frame 14 and is coupled to the rotary rack 21 for operating to rotate the rotary rack 21 so as to switch the grinding head 31 at the front end of the front frame 14. In the actual arrangement, the rotary rack 21 is provided with an operating lever 26 that located at the rotation axis of the rotary rack 21; the switch button 71 is rotatable and has a socket 76 coupled to the operating lever 2. When the switch button 71 rotates, the lever 76 is driven by the socket 76 to drive the rotary rack 21 to rotate. In this embodiment, the switch button 71 and the locking knob 61 are respectively located on both sides of the rotary rack 21 to avoid interference between the two structures.

The above explains the structure of the present invention. How the invention works is explained hereinafter.

Referring to FIGS. 9A and 9B and FIGS. 1-8 again, in the normal state, the locking rod 62 is located in one groove 23 to lock the rotary rack 21, and the rotating rack 21 has one grinding head 31 located at the front end of the front frame 14. When the user wants to use the present invention, the user needs only to switch on the present invention. When switched on, the driving device 41 drives the transmission mechanism 51 to rotate the grinding head 31. At this time, the user can put the rotating grinding head 31 against the dead skin or calluses to be worn away to carry out the grinding action.

Referring to FIGS. 10A, 10B and FIGS. 1-8 again, to switch the grinding head 31, simply move the locking knob 61 to the release position R where the rotary rack 21 is in a rotatable state. At this time, the user can operate the switch button 71 or directly rotate the rotary rack 21 to shift the desired grinding head 31 to the front end of the front frame 14, and then operate the locking knob 61 to the locking position. When the locking knob 61 is in the locking position, the locking rod 62 is engaged into one of the grooves 23 to lock the rotary rack 21. Then, you can use the multi head foot grinder as described above.

As can be seen from the above, the multi head foot grinder of the present invention has a plurality of grinding heads 31, which can be simply shifted and locked by the user, so that the user has a variety of grinding roughness options, and the user can save the inconvenience of saving and replacing the grinding heads 31.

What is claimed is:

1. A multi head foot grinder, comprising;
a housing comprising a grip and a front frame located on a front side of said grip;
a rotary rack rotatably mounted on said front frame;
a plurality of grinding heads, said grinding heads having an elongated shape, each said grinding head comprising a pivot that has two opposite ends thereof pivotally mounted in said rotary rack such that when said rotary rack is rotated, one said grinding head is shifted to a front end of said front frame;
a driving device mounted in said housing;
a transmission mechanism mounted on said front frame and said rotary rack and coupled to said driving device and the said grinding head that is shifted to the front end of said front frame, said transmission mechanism being drivable by said driving device to rotate the coupled said grinding head; and
a locking knob mounted on said front frame and movable between a locking position and a release position, said locking knob comprising a locking rod, said locking rod interfering with said rotary rack to prohibit said rotary rack from rotation when said locking knob is disposed in said locking position, said locking rod being released from said rotary rack for allowing said rotary rack to be rotated by an external force when said locking knob is disposed in said release position.

2. The multi head foot grinder as claimed in claim 1, further comprising a switch button mounted on said front frame and connected to said rotary rack, said switch button being operable to rotate said rotary rack in switching the said grinding head that is located at the front end of said front frame.

3. The multi head foot grinder as claimed in claim 2, wherein said rotary rack comprises an operating lever located at the rotation axis of said rotary rack; said switch button is rotatable and comprises a socket that is coupled to said operating lever, said socket driving said operating lever to rotate said rotary rack when said switch button is rotated.

4. The multi head foot grinder as claimed in claim 1, wherein said transmission mechanism consists of a plurality of gears.

5. The multi head foot grinder as claimed in claim 4, wherein each said grinding head further comprises a gear located on one end of the pivot thereof; said transmission mechanism uses one gear thereof to mesh with the gear of the said grinding head that is located at the front end of said front frame.

6. The multi head foot grinder as claimed in claim 1, wherein said rotary rack comprises a circular hole located on one side thereof; the rotation axis of said rotary rack is disposed in said circular hole; said rotary rack further comprises a plurality of grooves spaced around a border edge of said circular hole, said grooves being spatially connected to said circular hole, each said groove corresponding to one respective said grinding head; when said locking knob is disposed in said locking position, said locking rod is engaged into one said groove to interfere with said rotary rack and to prohibit said rotary rack from rotation; when said locking knob is disposed said release position, said locking rod is disposed in said circular hole and kept away from said grooves to release said rotary rack.

7. The multi head foot grinder as claimed in claim 6, wherein said locking rod comprises an accommodation space that accommodates at least one gear of said transmission mechanism.
